(19) United States
(12) Patent Application Publication
(10) Pub. No.: US 2014/0228786 A1
(43) Pub. Date: Aug. 14, 2014

(54) ABSORBENT BODY FOR THE THERAPEUTIC TREATMENT OF A WOUND BY MEANS OF NEGATIVE PRESSURE

(71) Applicant: Paul Hartmann AG, Heidenheim (DE)

(72) Inventors: Pierre Croizat, Herbrechtingen (DE); Axel Eckstein, Heidenheim (DE); Cornelia Wolf, Herbrechtingen (DE)

(73) Assignee: Paul Hartmann AG, Heidenheim (DE)

(21) Appl. No.: 14/136,431

(22) Filed: Dec. 20, 2013

Related U.S. Application Data
(60) Provisional application No. 61/750,496, filed on Jan. 9, 2013.

(30) Foreign Application Priority Data
Dec. 21, 2012 (DE) DE102012025125.4

(51) Int. Cl. A61M 1/00; A61F 13/00

(52) U.S. Cl. CPC A61M 1/0088; A61F 13/00068; A61F 2013/00536; A61F 2013/00748
USPC 604/319

(57) ABSTRACT

The invention relates in general to an absorbent for the therapeutic treatment of a wound on the human or animal body by means of negative pressure. More particularly, the invention relates to an absorbent for the therapeutic treatment by means of negative pressure of a wound comprising, at its surface, a tissue structure which is sensitive and lies deep in the intact body, for example a tendon or bone.

### CROSS REFERENCE TO RELATED APPLICATIONS

The present application claims priority to U.S. Provisional Application No. 61/750,496 filed Jan. 9, 2013 and to German Application No. DE 2012 025 125.4 filed Dec. 21, 2012, both of which applications are incorporated by reference herein.

### DESCRIPTION

The invention relates in general to an absorbent for the therapeutic treatment of a wound on the human or animal body by means of negative pressure. More particularly, the invention relates to an absorbent for the therapeutic treatment by means of negative pressure of a wound comprising, at its surface, a tissue structure which is sensitive and lies deep in the intact body, for example a tendon or bone.

A wound is understood to mean the break in the cohesion of tissues forming the outer covering of the body in humans or animals. It can be associated with substance loss.

Apparatuses for negative-pressure wound therapy are known in the prior art. For example, WO1993/009727 describes an apparatus for promoting wound healing by the application of negative pressure to the skin area which includes the wound and surrounds the wound. The apparatus according to WO1993/009727 comprises vacuum means for generating the negative pressure, an airtight wound covering, referred to as sealing means, which is in functional connection with the vacuum means, and also a wound dressing, referred to as screen means, for positioning on the wound within the sealing means. The screen means is an open-cell polymer foam, for example polyester foam. According to the description of WO1993/009727, the use of negative-pressure therapy can speed up wound healing for various types of wounds, for example burns, pressure sores or lacerations.

It is known from WO2011/003521 that absorbents which are charged with a solution and which comprise a superabsorbent polymer are suitable as wound dressing in negative-pressure wound therapy.

Apparatuses for negative-pressure therapy are commercially available, for example the Vivano® therapy system from the applicant Paul Hartmann AG (Germany). The commercially available apparatuses for negative-pressure wound therapy frequently use a wound dressing composed of an open-cell polymer foam. In practice, it has become apparent that wounds comprising, at their surface, tissue structures which are sensitive and lie deep in the intact body, for example tendons or bone, can be treated only unsatisfactorily with such apparatuses, since there is the risk of the bone and tendon tissue drying out.

In the prior art, apparatuses for negative-pressure therapy of tissue structures lying deep in the intact body are additionally known. For instance, WO2007/106590 discloses a negative-pressure therapy system for treating deep injuries on, for example, bone, tendons and ligaments, comprising a wound contact element, a suction hose and a flush hose. The wound contact element is surgically introduced into the body and is designed in such a way that it can be fixed to the tissue to be treated. The flexible wound contact element has openings and also either projections or an additional layer of open-pore material.

Similarly, WO2010/075313 is directed to a negative-pressure therapy system comprising a membrane which is contacted with the tissue to be treated with negative pressure. The membrane side lying on the tissue to be treated has a multiplicity of projections which form at least one channel which transfers the negative pressure to the tissue. The system is preferably used for treating bone tissue, with the preferably flexible membrane which is degradable by the body and to which the suction hose is connected covering the bone at least in part.

In contrast, WO2010/078166 discloses a negative-pressure therapy system comprising a hose intended for placement on subcutaneous tissue, for example bone. A drainage or suction apparatus is introduced into the lumen of the hose. Both the hose and the drainage or suction apparatus have openings at their ends situated in the body of the patient, and so negative pressure can be passed on onto the adjacent tissue. This setup allows simple removal of the drainage or suction apparatus from the body of the patient in the event that the apparatus needs to be cleaned or exchanged.

The systems known from WO2007/106590, WO2010/075313 and WO2010/078166 are thus primarily intended for applying negative pressure in a targeted manner to a subcutaneous tissue defect, the outer covering of the body covering the tissue defect being substantially intact. By contrast, they appear less suited for use on wounds which are associated with a loss of substance from the outer covering of the body or the skin tissue and in which tissue structures which are sensitive and lie deep in the intact body, for example tendons or bone, are exposed.

It is an object of the present invention to further improve negative-pressure wound therapy and to overcome the disadvantages in the prior art. More particularly, it is an object of the present invention to provide an improved wound dressing for the negative-pressure therapy of wounds which comprise, at their surface, tissue structures which are sensitive and lie deep in the intact body, for example tendons or bone. The invention achieves the object with an absorbent having the features described herein or with an apparatus for negative-pressure wound therapy having the features described herein.

It was found that, surprisingly, an absorbent comprising a superabsorbent polymer is suitable as a wound dressing for the negative-pressure therapy of a wound comprising, at its surface, tissue structures which are sensitive and lie deep in the intact body, for example tendons or bone. In this connection, the absorbent is contacted with the sensitive tissue structure during the negative-pressure therapy, and so the structure is protected by the absorbent until it is overlaid by newly formed granulation tissue. The protective action and the success of therapy is especially pronounced when the absorbent is charged with a solution. In this particularly preferred embodiment, the absorbent is always charged prior to the wound therapy, for example by the manufacturer or immediately prior to the therapy by the user.

An advantage of the absorbent charged with a solution is that, immediately after the start of the therapy, the tissue structure which is sensitive and lies deep in the intact body is kept in a moist environment and does not dry out owing to release of the charge solution, and so the tissue structure does not die during the negative-pressure therapy and rapid and successful wound healing is promoted. Further advantageous properties of the absorbent charged with a solution are its soft structure and its low tendency to adhere to and/or to grow together with the wound surface. As a result, the tissue structures which are sensitive and lie deep in the intact body are treated gently and the pain for the patient due to the negative-pressure therapy is reduced. Because the absorbent absorbs any wound exudate in exchange for released charge solution and takes up bacteria present in the wound exudate and other harmful constituents, wound infections can be counteracted and wound healing is further favored.

In a first aspect, the invention provides an absorbent comprising a superabsorbent polymer, for the therapeutic treatment by means of negative pressure of a wound comprising, at its surface, a tissue structure which is sensitive and lies deep in the intact body, the absorbent preferably being charged with a solution, more particularly Ringer's solution. In other words, in said first aspect, the invention comprises a product for the therapeutic treatment of a wound, the product being an absorbent comprising a superabsorbent polymer and the product being used for application in the negative-pressure therapy of a wound comprising, at its surface, a tissue structure which is sensitive and lies deep in the intact body. The product is preferably charged with a solution, more particularly Ringer's solution.

In a second aspect, the invention comprises an absorbent comprising a superabsorbent polymer, for the therapeutic treatment of a wound by means of negative pressure, wherein the absorbent is contacted, during the treatment, with a tissue structure at the surface of the wound, which structure is sensitive and lies deep in the intact body, in order to protect the tissue structure from drying out, and the absorbent preferably being charged with a solution, more particularly Ringer's solution. In other words, in said second aspect, the invention comprises a product for the therapeutic treatment of a wound, the product being an absorbent comprising a superabsorbent polymer and the product being contacted, during negative-pressure therapy, with a tissue structure at the surface of the wound, which structure is sensitive and lies deep in the intact body, in order to protect the tissue structure from drying out. The product is preferably charged with a solution, more particularly Ringer's solution.

On the basis of the abovementioned second aspect of the invention, the invention likewise comprises an absorbent comprising a superabsorbent polymer, for the therapeutic treatment of a wound by means of negative pressure, the absorbent being intended, during the treatment, to be contacted with a tissue structure at the surface of the wound, which structure is sensitive and lies deep in the intact body, in order to protect the tissue structure from drying out, and the absorbent preferably being charged with a solution, more particularly Ringer's solution. In other words, the invention therefore comprises a product for the therapeutic treatment of a wound, the product being an absorbent comprising a superabsorbent polymer and the product being intended, during negative-pressure therapy, to be contacted with a tissue structure at the surface of the wound, which structure is sensitive and lies deep in the intact body, in order to protect the tissue structure from drying out. The product is preferably charged with a solution, more particularly Ringer's solution.

In a third aspect, the invention comprises an absorbent comprising a superabsorbent polymer, for the therapeutic treatment of a wound by means of negative pressure, the absorbent being used, during the treatment, to keep moist a tissue structure at the surface of the wound, which structure is sensitive and lies deep in the intact body, in order to protect the tissue structure from drying out, and the absorbent preferably being charged with a solution, more particularly Ringer's solution. In other words, in said third aspect, the invention comprises a product for the therapeutic treatment of a wound, the product being an absorbent comprising a superabsorbent polymer and the product being used, during negative-pressure therapy, to keep moist a tissue structure at the surface of the wound, which structure is sensitive and lies deep in the intact body, in order to protect the tissue structure from drying out. The product is preferably charged with a solution, more particularly Ringer's solution.

In a fourth aspect, the invention comprises an apparatus for negative-pressure wound therapy. The apparatus comprises an airtight cover material which is used to seal the wound airtight. The apparatus further comprises means for the functional connection of the wound space to a negative-pressure source situated outside the cover material. This makes it possible to produce negative pressure in the wound space and to aspirate liquids from the wound space. The apparatus further comprises at least one absorbent or at least one product according to any of the aforementioned aspects of the invention, which is introducible or is introduced into the space between the surface of the wound and the cover material.

As a further aspect, the present invention includes a method for application in negative-pressure wound therapy. Said method comprises the steps of
a) introducing an absorbent comprising a superabsorbent polymer into a wound comprising, at its surface, a tissue structure which is sensitive and lies deep in the intact body,
b) sealing the wound by means of an airtight cover material, forming a wound space comprising the absorbent between the surface of the wound and the cover material,
c) attaching means for the functional connection of the wound space to a negative-pressure source situated outside the cover material, making it possible to produce negative pressure in the wound space and to aspirate liquids from the wound space,
   the absorbent preferably being charged with a solution, more particularly Ringer's solution, and preferably being contacted with the tissue structure at the surface of the wound, which structure is sensitive and lies deep in the intact body.

In a preferred embodiment, the method comprises a further step for application in negative-pressure wound therapy, in which step a pressure-distribution layer is introduced into the wound. In said preferred embodiment, the method comprises altogether the following steps:
a) introducing an absorbent comprising a superabsorbent polymer into a wound comprising, at its surface, a tissue structure which is sensitive and lies deep in the intact body,
b) introducing a pressure-distribution layer, preferably a pressure-distribution layer comprising a foam, particularly preferably a pressure-distribution layer comprising an open-cell polyurethane foam, into the wound,
c) sealing the wound by means of an airtight cover material, forming a wound space comprising the absorbent and the pressure-distribution layer between the surface of the wound and the cover material,
d) attaching means for the functional connection of the wound space to a negative-pressure source situated outside the cover material, making it possible to produce negative pressure in the wound space and to aspirate liquids from the wound space,
   the absorbent preferably being charged with a solution, more particularly Ringer's solution, and preferably being contacted with the tissue structure at the surface of the wound, which structure is sensitive and lies deep in the intact body, and
   the pressure-distribution layer preferably being arranged between the absorbent and the cover material.

The aforementioned methods can be followed by a further method for application in negative-pressure wound therapy, comprising the following steps:
a) introducing a wound dressing, preferably a wound dressing comprising a foam, particularly preferably a wound dressing comprising an open-cell polyurethane foam, into a wound,
b) sealing the wound by means of an airtight cover material, forming a wound space comprising the wound dressing between the surface of the wound and the cover material,
c) attaching means for the functional connection of the wound space to a negative-pressure source situated outside the cover material, making it possible to produce negative pressure in the wound space and to aspirate liquids from the wound space,
   the wound comprising, at its surface, preferably no tissue structure which is sensitive and lies deep in the intact body. The method can optionally comprise a split-thickness skin graft.

The absorbent according to the invention comprises a superabsorbent polymer. A superabsorbent polymer is understood in general to mean a water-insoluble swellable polymer which can take up many times its own weight of liquid, for example water, salt solutions or body fluids. The liquid uptake leads to the formation of a hydrogel. The uptake capability for pure water is typically higher than the uptake capability for salt-containing liquid. In the context of this invention, the expression “superabsorbent polymer” is understood in particular to mean a polymer which, according to the standard test method WSP 240.2 (05), has a w value (free swell capacity) of at least 10 g/g, preferably at least 20 g/g. The test method WSP 240.2 (05) for determining the w value is described in “Standard Test Methods for the Nonwovens and Related Industries”, edition 2008 (published by “EDANA, International Association Serving the Nonwovens and Related Industries”, Cary, N.C., USA and “INDA, Association of the Nonwovens Fabrics Industry”, Brussels, Belgium). WSP 240.2 (05) according to EDANA is a standard test method for determining the w value (free swell capacity) of superabsorbent polyacrylate powder. According to WSP 240.2 (05), the free uptake capacity is determined for a 0.9 percent by weight saline solution. In conjunction with the present invention, the determination of the w value of superabsorbent material which is not polyacrylate powder is carried out in a corresponding manner.

The superabsorbent polymer may preferably be present in the form of particles or fibers.

Possible wounds for the present invention are wounds on the human or animal body which comprise one or more tissue structures which are sensitive and lie deep in the intact body. In conjunction with the present invention, sensitive means that the tissue structure is exposed, during the negative-pressure therapy, to a particularly high risk of drying out, as a consequence of which the tissue structure can die. This is especially the case when the tissue structure comprises no blood vessels or only a few blood vessels and the substance exchange of the tissue structure or the substance exchange of parts of the tissue structure thus substantially involves diffusion processes. In addition, “lying deep in the intact body” is to be understood to mean that the tissue structure is located in or below the subcutis in the intact or nonwounded state of the human or animal body.

The tissue structures which are sensitive and lie deep in the intact body are found, for example owing to an injury or a surgical procedure, at the surface of the wound and are not covered by a further tissue layer, and in conjunction with the present invention this is also referred to as being exposed. Wounds having tissue structures at the surface, which structures are sensitive and lie deep in the intact body, can, for example, be caused by mechanical trauma, by burns, by a metabolic or circulation disorder, or by a pressure ulcer.

The absorbent according to the invention is intended to protect the tissue structures which are sensitive and lie deep in the intact body from drying out during the negative-pressure therapy by keeping them moist or, synonymously, by keeping them in a moist environment.

The absorbent according to the invention is particularly suited to keeping moist the tissue structures which are sensitive and lie deep in the intact body when it has been charged with a solution.

The absorbent according to the invention is preferably contacted indirectly or directly with the tissue structure which is sensitive and lies deep in the intact body. Contact can be indirect when a wound contact layer which does not impair or only negligibly impairs the generation of the moist environment is introduced between the tissue structure which is sensitive and lies deep in the intact body and the absorbent. Accordingly, when using a wound contact layer, it has to be ensured that the absorbent according to the invention can keep in a moist environment the tissue structure which is sensitive and lies deep in the intact body. In addition, it is advantageous when the tissue structure to be protected from drying out is completely covered by the absorbent. Depending on the size of the tissue structure to be treated in relation to the size of the absorbent, one absorbent may be sufficient for this purpose or multiple absorbents have to be used. Multiple absorbents are possibly also necessary when multiple tissue structures which are sensitive and lie deep in the intact body are present in different areas of the wound surface, all of which are to be contacted with an absorbent.

Preferably, the tissue structure which is sensitive and lies deep in the intact body is substantially covered with the absorbent according to the invention. A pressure-distribution layer can additionally be introduced between wound surface and cover material. However, it is also possible for the wound space, i.e. the space present between wound surface and cover material, to be solely filled by one or more absorbents.

In a preferred embodiment of the invention, the tissue structure which is sensitive and lies deep in the intact body is in particular a ligament, a tendon, cartilage and/or bone. Here, the aforementioned tissue structure which is sensitive and lies deep in the intact body need not be completely exposed. In most cases, the tissue structure is instead only partly exposed. Particularly preferably, the tissue structure which is sensitive and lies deep in the intact body does not have any defects or tissue damage. A defect or tissue damage is understood in this connection to mean the absence of part of the tissue structure which is sensitive and lies deep in the intact body.

With regard to whether the tissue structure at the surface of the wound, which structure is sensitive and lies deep in the intact body, needs to be protected from drying out during the negative-pressure therapy by means of the absorbent according to the invention, this can be assessed by the attending physician, generally a physician with experience in the field of wound treatment, usually on the basis of the particular wound situation. Criteria to be considered here are, for example, the amount of exudate released by the wound and the proportion or the size of the exposed tissue structure. The tissue structure which is sensitive and lies deep in the intact body may be at particular risk of drying out when the tissue structure occupies an area of at least 1 cm², for example at least 2 cm² or at least 3 cm². If the attending physician wishes to approximately determine the area of the tissue structure at the wound surface, which structure is sensitive and lies deep in the intact body, it is possible to apply an appropriate sheet containing a square grid to the wound surface. The outline of the tissue structure which is sensitive and lies deep in the intact body is transferred to the sheet and the area thereof is determined by means of the method of counting the number of intersection points. The method of counting the number of intersection points is described in detail in the article “Was nicht dokumentiert wurde, gilt als nicht erbracht” (“There is no evidence without documentation”) (Hoppe et al., Die Schwester Der Pfleger, 45th year, November 2006).

To protect the tissue structure which is sensitive and lies deep in the intact body from drying out, the absorbent is contacted with the tissue structure preferably for at least 6 hours, particularly preferably for at least 1 day, very particularly preferably for at least 2 days. Generally, the absorbent according to the invention is contacted with the tissue structure which is sensitive and lies deep in the intact body for not longer than 4 days, preferably for not longer than 3 days. Thereafter, the absorbent can be exchanged and the negative-pressure therapy continued, preferably until the tissue structure which is sensitive and lies deep in the intact body is no longer exposed in the wound, i.e. is no longer situated at the surface of the wound. Until this time, repeated replacement of the absorbent may be required. As soon as the tissue structure which is sensitive and lies deep in the intact body is no longer present at the surface of the wound, but is instead covered by newly formed tissue, the negative-pressure therapy—possibly with the use of other wound dressings and supplementary surgical measures such as, for example, a split-thickness skin graft—can be continued. It is also possible to follow the method for negative-pressure therapy, as proposed here, with further wound treatment methods customary in medicine without the use of negative pressure. A combination of medically appropriate follow-up therapies is likewise possible.

Appropriate follow-up therapies are known from the prior art to a person skilled in the art, for example from WO1993/009727, which has already been mentioned. An appropriate follow-up therapy can also consist in using, as wound dressing, an absorbent as used in the present invention without the use of negative pressure. Such follow-up therapy is described in the European patent applications with the application numbers 12006687.3 and 12006688.1, which are not yet published.

In a preferred embodiment of the invention, the superabsorbent polymer is a superabsorbent polyacrylate. Here, in the context of the present invention, polyacrylate is understood to mean a synthetic polymer comprising, as monomer (M1), acrylic acid (2-propenoic acid, CH₂═CH—CO₂H) and/or a salt thereof. The monomer content is in particular more than percent by weight of acrylic acid and/or a salt thereof (based on the total weight of the polyacrylate). Polyacrylates according to the invention preferably have a monomer content of more than 80 percent by weight of acrylic acid and/or a salt thereof and very particularly preferably more than 95 percent by weight of acrylic acid and/or a salt thereof, based on the total weight of the polyacrylate. The polyacrylate can be present as a homopolymer, copolymer or block polymer. If the polyacrylate is present as a copolymer or block polymer, the monomer content of the monomer M1 in the polymer is always more than 70 percent by weight, more particularly more than 80 percent by weight, and very particularly preferably more than 95 percent by weight, based on the total weight of the polyacrylate. These copolymer polyacrylates or block-polymer polyacrylates can contain, in addition to the monomer M1, in particular α,β-unsaturated ethers (vinyl ethers), α,β-unsaturated carboxylic acids or α,β-unsaturated carboxylic esters (vinyl esters) as comonomers M2. Of the comonomers M2 of α,β-unsaturated carboxylic acids, particular preference is given to methacrylic acid (2-methylpropenoic acid), ethacrylic acid (2-ethylpropenoic acid), crotonic acid (2-butenoic acid), sorbic acid (trans,trans-2,4-hexadienoic acid), maleic acid (cis-2-butenedioic acid) or fumaric acid (trans-2-butenedioic acid). However, it is also possible for the polyacrylate to consist of a) a homopolymer composed of acrylic acid and/or b) a copolymer composed of i) acrylic acid and a salt of the acrylic acid, ii) composed of methacrylic acid and a salt of the methacrylic acid or iii) composed of acrylic acid and methacrylic acid and the salts thereof. In addition, it is, however, also possible for the polyacrylate to be a mixture of different polyacrylates.

To achieve, in the absorbent, a pH advantageous for the wound treatment in the range from about pH 4.0 to pH 8.5, more particularly between pH 5.0 and 6.0, the polyacrylate is preferably present as a partially neutralized polymer; more particularly, the degree of neutralization should be between 20% and 90%, particularly preferably between 45% and 80%.

In addition, it has become apparent that particularly advantageous effects for wound healing can arise when the absorbent comprises a mixture of polyacrylate particles, the particle mixture containing polyacrylate particles of varying size, characterized in that the particle mixture contains

a) 5 to 100% by weight, preferably 5 to 98% by weight, of particles having a particle size x where x<300 μm, and
b) 0 to 95% by weight, preferably 2 to 95% by weight, of particles having a particle size x where x>300 μm

With respect to further polyacrylates and mixtures of polyacrylate particles usable for the present invention in a particularly advantageous manner, reference is made to the polyacrylates and particle mixtures disclosed in WO2009/068249.

In conjunction with the present invention, the particle size is determined analogously to EDANA 420.2-02, with the screens (diameter 200 mm) having hole sizes meeting the specifications. Furthermore, it is also possible to use screens having different hole sizes, for example 125 μm, 160 μm, 630 μm, 900 μm, and 1500 μm. Here, dry polyacrylate particles having a moisture content of less than 10% by weight of water, based on the total weight of the particles, are taken as a basis, with the moisture content being determined according to EDANA 450.2-02.

In addition to the superabsorbent polymer, which is preferably a superabsorbent polyacrylate, the absorbent can comprise a support material, the support material comprising a hydrophilic fibrous material. Here, the hydrophilic fibrous material used can be in particular water-insoluble fibers composed of cellulose, more particularly largely delignificated industrial pulp fibers, more particularly wood pulp fibers, more particularly with a fiber length of <5 mm. The fibrous material can also contain hydrophilic fibrous material composed of regenerated cellulose, carboxymethylcellulose, carboxyethylcellulose, hydroxymethylcellulose or hydroxyethylcellulose. It is also possible to have a fiber mixture composed of cellulose fibers, regenerated cellulose fibers, carboxymethylcellulose fibers, carboxyethylcellulose fibers, hydroxymethylcellulose fibers or hydroxyethylcellulose fibers and fibers composed of polyethylene, polypropylene or polyester.

In a preferred embodiment of the invention, the absorbent comprises a superabsorbent polymer and, as support material, a mixture of cellulose fibers and polypropylene fibers. These fibers can be processed together with the superabsorbent polymer to form a layer in a so-called airlaid method.

In addition, in a preferred embodiment of the invention, the absorbent is an absorbent surrounded by a textile shell. The shell is permeable to liquid at least in some areas and the superabsorbent polymer is arranged therein. In other words, the superabsorbent polymer is surrounded by the shell. The shell can consist of a single material or comprise multiple materials. The shell can in particular consist of an interlock knit composed of polypropylene fibers.

In a particularly preferred embodiment of the invention, the absorbent is an absorbent comprising a textile shell composed of an interlock knit composed of polypropylene fibers, in which shell a superabsorbent polyacrylate, cellulose fibers and polypropylene fibers are arranged. The superabsorbent polyacrylate particles and the cellulose fibers and polypropylene fibers within the shell are processed to form a layer according to an airlaid method. Such a product is commercially available under the name TenderWet® (Paul Hartmann AG, Germany).

A particularly suitable soft and plastically deformable absorbent is obtained in particular when its shell at least partly consists of a textile surface which is nonelastically stretchable in the longitudinal, transverse and diagonal direction. In this connection, reference may be made to EP0594034B1. One of a plurality of suitable methods for welding shells composed of thermoplastics is ultrasonic welding. The absorbent can, for example, have a round, square or oval shape. However, other shapes are also conceivable.

The absorbent can have different amounts of superabsorbent polymer and support material. Preferably, the absorbent contains at least 10 percent by weight of polyacrylate (based on the support material). However, particular preference is given to absorbents comprising at least 20 percent by weight, more particularly at least 25 percent by weight, and very particularly preferably at least 30 percent by weight of polyacrylate (based on the support material). So that the absorbent is not restricted with respect to the uptake and release of aqueous liquids, it should be ensured, however, that the polyacrylate content with respect to the support material is preferably not more than 90 percent by weight and more particularly not more than 75 percent by weight.

In a preferred embodiment of the invention, the absorbent is charged with a solution. Charging is effected preferably until saturation and preferably with an aqueous solution which can swell the superabsorbent polymer and bring it to a gelatinous state. The aqueous solution is preferably salt-containing. In addition, the aqueous solution is a synthetic solution, and this is to be understood to mean that the solution is prepared technically and does not comprise body fluids or fluids discharged from the body, for example wound exudate. The solution used for charging can be released by the absorbent during the negative-pressure therapy, with the solution being irreversibly removed from the wound space only in part owing to the applied negative pressure. In exchange for released charge solution, the absorbent can absorb any wound exudate and bacteria present therein and take up other harmful constituents, counteracting wound infections and further favoring wound healing.

In a further preferred embodiment of the invention, the absorbent is charged with at least 500 percent by weight, based on the weight of the dry absorbent, of the solution. The absorbent can, for example, be charged with at least 600 percent by weight, at least 800 percent by weight or at least 1000 percent by weight of an aqueous solution, based on the weight of the dry absorbent. In addition, the absorbent is preferably charged with less than 5000 percent by weight, for example less than 3500 percent by weight, for example less than 2500 percent by weight, of an aqueous solution.

In a particularly preferred embodiment of the invention, the absorbent contains that amount of an aqueous solution which corresponds to the maximum uptake capacity of the absorbent for Ringer's solution. The maximum uptake capacity for Ringer's solution can be determined according to the aforementioned test method WSP 240.2 (05), but with a) Ringer's solution being used instead of the saline solution used in WSP 240.2 (05) and b) the absorbent according to the invention being used instead of the test substance sealed within a shell (“bag” according to section 6.1 of WSP 240.2 (05)). The maximum uptake capacity corresponds to the weight difference, as determined gravimetrically according to this method, between dry absorbent and charged absorbent, with a deviation of the weight difference by 15% in the upward or downward direction being encompassed.

Preferably, the aqueous solution contains more than 50, for example more than 70, more than 80, more than 90 or 100 percent by volume of water. It can contain at least 5 mmol/l of sodium ions, at least 0.1 mmol/l of potassium ions, at least 0.1 mmol/l of calcium ions and/or at least 5 mmol/l of chloride ions. Optionally, the aqueous solution contains further inorganic cations and/or anions, optionally organic anions, and optionally additives of bioorganic compounds. The pH is preferably from 4 to 9. The viscosity at 20° C. is preferably between 0.8 mPa·s and 150 mPa·s. The viscosity of the solution is determined using a Brookfield viscometer (unit: 1 Pa·s=1 Ns/m²).

In a particularly preferred embodiment of the invention, the solution is a synthetic, aqueous solution, more particularly Ringer's solution. A Ringer's solution is understood to mean a synthetic solution which is approximately iso-osmotic relative to blood and which comprises sodium chloride, potassium chloride and calcium chloride dissolved in distilled water. Preferably, the Ringer's solution contains 147 mmol/l of sodium ions, 4.0 mmol/l of potassium ions, 3.0 mmol/l of calcium ions and 157 mmol/l of chloride ions, with a deviation of the particular ion concentration from the specified value by 5% being possible.

In a further embodiment of the invention, the absorbent can comprise an antimicrobial substance, for example as an alternative or in addition to the above-described aqueous solution. Preferably, the antimicrobial substance is or comprises a substance which is present in cationic form at pH 4-7.5 and has antimicrobial action, for example substances having amino or imino groups. The cationic antimicrobial substance can be antimicrobial metal cations, more particularly silver cations, for example a complex of 1-vinyl-2-pyrrolidone with silver cations. Particularly suitable cationic substances having antimicrobial action are biguanide derivatives such as chlorhexidine or polybiguanides, such as polyethylene biguanide (PEB), polytetramethylene biguanide (PTMB) or polyethylene hexamethylene biguanides (PEHMB). A particularly preferred polybiguanide is polyhexamethylene biguanide (PHMB, or polyhexanide). Further suitable cationic substances having antimicrobial action are polyguanidines, for example polyhexamethylene guanidines (PHMG), N-octyl-1-[10-(4-octyliminopyridin-1-yl)decyl]pyridin-4-imine (octenidine), quaternary ammonium compounds, for example benzalkonium chloride or cetylpyridinium chloride, triazines, for example 1-(3-chloroallyl)-3,5,7-triaza-1-azoniaadamantane chloride, or the ammonium compound taurolidine.

The apparatus according to the invention comprises a cover material for airtight sealing of the wound. “Airtight sealing” is not to be understood here to mean that there is no gas exchange at all between the wound space and its environment. On the contrary, “airtight sealing” in this connection means that, taking into account the negative-pressure pump used, the negative pressure required for negative-pressure wound therapy can be sustained. It is therefore also possible to use cover materials which have negligible gas permeability, provided that the negative pressure required for the negative-pressure therapy can be sustained.

The airtight cover material can, for example, be present in the form of a shell consisting of a solid material or in the form of a flexible film. Combinations of solid frames or base plates with flexible films are also conceivable. In a preferred embodiment of the invention, the cover material comprises a water-insoluble polymer, or a metal foil, for airtight sealing of the wound.

In a particularly preferred embodiment of the invention, the water-insoluble polymer is polyurethane, polyester, polypropylene, polyethylene, polyamide or polyvinyl chloride, polyorganosiloxane (silicone), or a mixture thereof. Further suitable polymeric film materials are known to a person skilled in the art.

It is also possible to use, as cover material, finished products which have the abovementioned properties. Polyurethane film with the brand name Hydrofilm® (Paul Hartmann AG, Germany) or Visulin® (Paul Hartmann AG, Germany) has been found to be a particularly suitable cover material for the apparatus according to the invention.

The cover material is fastened in the wound surrounding area or on the wound edge in such a way that an airtight wound seal is ensured. For this purpose, it may be useful for the cover material to be self-adhesive over its entire surface or to have a self-adhesive edge. Alternatively, fastening and sealing can, for example, be carried out using an adhesive film, using a liquid adhesive or using a sealing compound. However, another possibility is for the cover material to be held on the wound merely by means of the negative pressure generated during the negative-pressure treatment.

In a preferred embodiment of the invention, the cover material comprises a film composed of one or more water-insoluble polymers, the film being self-adhesive over its entire surface or having a self-adhesive edge.

The apparatus according to the invention for negative-pressure wound therapy additionally comprises means for the functional connection of the wound space to a negative-pressure source situated outside the cover material, making it possible to produce negative pressure in the wound space and to aspirate liquids from the wound space.

In conjunction with the invention, the expression “negative pressure in the wound space” refers to air pressure lowered within the wound bandage with respect to the environmental air pressure (atmospheric air pressure). “Within the wound bandage” is understood to mean the space formed between the cover material and the wound. “Negative pressure” is frequently also referred to as “reduced pressure”.

In conjunction with the invention, the pressure difference between the air pressure within the wound bandage and the environmental air pressure is reported in mmHg (millimeter of mercury), since this is customary in the area of negative-pressure therapy. 1 mmHg corresponds to one Torr or 133.322 Pa (pascal). In conjunction with the invention, the negative pressure, i.e. the pressure difference between the air pressure within the wound bandage and the environmental air pressure, is reported as a positive numerical value in mmHg.

In one embodiment of the invention, the negative pressure is negative pressure of no more than 250 mmHg. This negative-pressure range of no more than 250 mmHg has been found to be suitable for wound healing. In a preferred embodiment of the invention, the negative pressure is negative pressure of at least 10 mmHg and no more than 150 mmHg. As is well known in the prior art, the negative pressure can be negative pressure which is constant or varies over time.

The functional connection can, for example, be established using a connecting line or using a negative-pressure connecting piece. Negative-pressure connecting pieces are also known by the term “port” to a person skilled in the art.

In one embodiment of the invention, the means for the functional connection of the wound space to a negative-pressure source situated outside the cover material is at least one connecting line. The at least one connecting line can be passed through the cover material. Alternatively, the at least one connecting line can be passed through under the edge of the cover material. In both cases, the site of passage must be sealed airtight so that the desired negative pressure in the bandage can be sustained. Suitable sealants are, for example, an adhesive film, an adhesive compound, or an adhesive strip.

The connecting line can be, for example, a hose, a tube or another body having a hollow space. A suitable hose is, for example, a silicone drainage hose.

It is useful for the connecting line to have a negative-pressure adapter at the end situated outside the wound bandage, for connectability with the further components of the negative-pressure system. The connecting line has an opening at the end situated within the wound bandage.

In a further embodiment of the invention, the means for the functional connection of the wound space to a negative-pressure source situated outside the cover material is a negative-pressure connecting piece (port) which can be fastened to either the inner or the outer side of the cover material, the cover material having suitable openings. In this embodiment, an airtight seal of either the passage opening (port inside) or the supporting surface (port outside) must also be ensured. The seal can, for example, be established using an adhesive film, using an adhesive compound, or using an adhesive strip. It is also conceivable for the port itself to have corresponding fastening means, for example adhesive surfaces.

Suitable negative-pressure connecting pieces are commercially available. They are typically negative-pressure connecting pieces which are fastened to the outer side of the cover material. It is useful for the negative-pressure connecting piece to also have a negative-pressure adapter, for connectability with the further components of the negative-pressure system.

In a preferred embodiment of the invention, cover material and the means for the functional connection of the wound space to a negative-pressure source situated outside the cover material are already provided ready-to-use connected to one another. It is very particularly preferred for this embodiment to contain a film composed of one or more water-insoluble polymers and having a self-adhesive edge, since this arrangement substantially facilitates the application of the bandage.

In a preferred embodiment of the invention, the apparatus for negative-pressure therapy additionally comprises at least one pressure-distribution layer which is introducible or is introduced between the absorbent and the cover material. The advantage of an additional pressure-distribution layer can consist in the pressure exerted by the bandage on the wound base being even more uniformly distributable as a result of the use of the pressure-distribution layer. In addition, the pressure-distribution layer can store additional wound exudate or further improve the aspiration of the wound exudate from the wound space.

The additional pressure-distribution layer can consist of an open-cell or semi-open-cell foam, a spacer knit, a textile layer, a structured gel, or a permeable nonwoven layer. The open-cell or semi-open-cell foam can be polyvinyl alcohol foam, silicone foam or polyurethane foam. Suitable textile layers are, inter alia, gauze swabs with cut edges folded in or lattice tulles. The additional press-distribution layer can be designed in such a way that liquid such as wound exudate is passed therethrough. To this end, the pressure-distribution layer can contain suitable channels or a suitable opening, or consist of a material permeable to liquids. The additional pressure-distribution layer can be connected adhesively or nonadhesively to the absorbent. It can also be separated from the absorbent by a further layer, for example one ply of a textile layer.

In a preferred embodiment of the invention, the apparatus for negative-pressure therapy additionally comprises at least one wound contact layer which is introducible and/or is introduced between the surface of the wound and the pressure-distribution layer and/or between the surface of the wound and the absorbent. The additional wound contact layer can be connected adhesively or nonadhesively to the absorbent or to the pressure-distribution layer. The apparatus for negative-pressure therapy can also comprise multiple wound contact layers composed optionally of different materials.

The wound contact layer can, in principle, be any wound contact layer known from the prior art, provided that, firstly, passage of liquids through the wound contact layer is ensured and, secondly, the material shows no tendency to grow together with or to adhere to the wound tissue. The wound contact layer can, for example, be an open-cell or semi-open-cell foam, more particularly polyurethane, a hydrocolloid, a structured gel, a polyorganosiloxane (silicone), a permeable nonwoven layer or a lattice tulle. The permeable nonwoven layer or lattice tulle preferably consists of a hydrophobic material, for example polyester or polyamide, and can additionally be provided with an ointment. The wound contact layer can also consist of a bioresorbable material as known from DE19609551 or from WO02/072163. Another possibility is that the wound contact layer comprises an antimicrobial coating, for example a silver coating, or wound healing-promoting substances such as growth factors. Suitable, commercially available wound contact layers are, for example, the ointment dressings with the brand names Hydrotüll®, Atrauman® and Atrauman Ag® (Paul Hartmann AG, Germany).

In a particularly preferred embodiment of the invention, the apparatus for negative-pressure therapy additionally comprises at least one wound contact layer which is introduced between the surface of the wound and the pressure-distribution layer.

### FIGURES

The absorbent according to the invention and the apparatus according to the invention for negative-pressure wound therapy will be more particularly elucidated below using drawings. However, the invention is not to be understood to be reduced to the embodiments shown in the drawings or in the description of the drawings. On the contrary, the invention also encompasses combinations of the individual features of the alternative forms.

FIG. 1 shows one embodiment of the apparatus according to the invention for negative-pressure wound therapy (diagrammatical side view).

FIG. 2 shows a further embodiment of the apparatus according to the invention for negative-pressure wound therapy, the apparatus comprising a pressure-distribution layer (diagrammatical side view).

FIG. 3 shows a further embodiment of the apparatus according to the invention for negative-pressure wound therapy, the apparatus comprising a pressure-distribution layer (diagrammatical side view).

FIG. 4 shows a further embodiment of the apparatus according to the invention for negative-pressure wound therapy, the apparatus comprising a pressure-distribution layer and a wound contact layer (diagrammatical side view).

FIG. 5 shows a further embodiment of the apparatus according to the invention for negative-pressure wound therapy, the apparatus comprising a pressure-distribution layer and a wound contact layer (diagrammatical side view).

### FIGURE LEGEND

1 airtight cover material
2 surface of the wound
3 tissue structure which is sensitive and lies deep in the intact body
4 absorbent comprising a superabsorbent polymer
5 wound space
6 wound surrounding area
7 negative-pressure connecting piece (port)
8 connecting line (hose)
9 collection vessel
10 negative-pressure unit
11 pressure-distribution layer
12 wound contact layer

### DESCRIPTION OF THE FIGURES

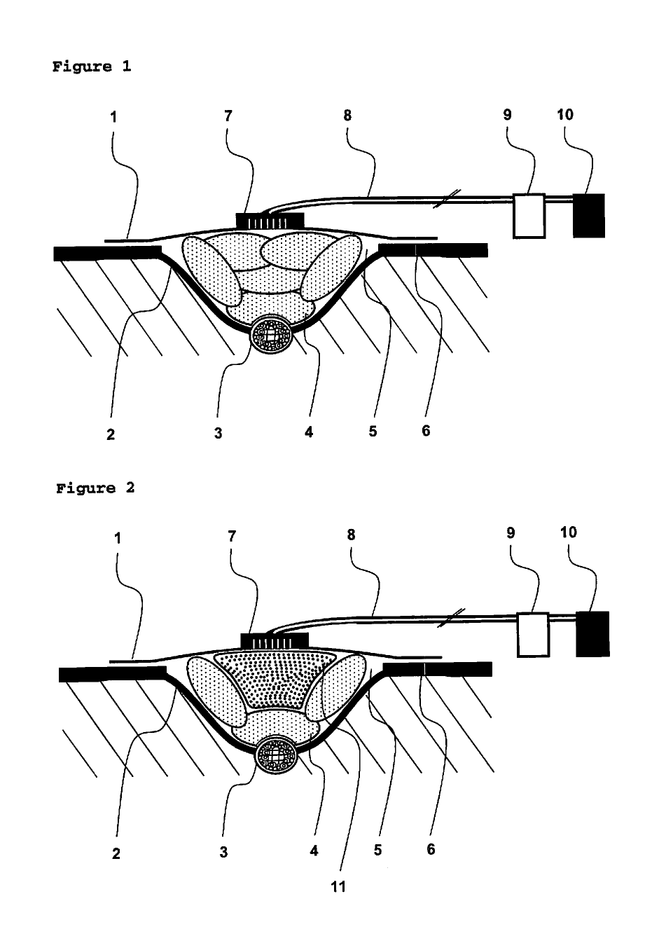

FIG. 1 shows one embodiment of the apparatus according to the invention for negative-pressure wound therapy. The apparatus comprises an airtight cover material (1) for airtight sealing of the wound and of the wound surrounding area (6), means (7) for the functional connection of the wound space (5) to a negative-pressure source (10) situated outside the cover material (1), and multiple absorbents (4) comprising a superabsorbent polymer. The wound comprises at its surface (2), as tissue structure which is sensitive and lies deep in the intact body, a section of bone (3) which has been contacted with one of the absorbents (4). The absorbents (4) should be inserted into the wound space (5) in such a way that as few cavities as possible are produced and good contact with the wound surface (2) and especially the exposed bone section (3) is ensured. In this embodiment, the number of absorbents (4) required for this purpose is substantially determined by the size of the wound space (5).

The cover material (1) is fastened in the region of the wound surrounding area (6), which usually has intact skin. The size of the cover material should be measured in such a way that the cover material can be fastened outside the wound region in the region of the wound surrounding area (6). The cover material (1) can be of different dimensions and shapes, for example circular, oval or rectangular. It can also be of an irregular shape adapted to the wound. The cover material (1) can be an opaque material, a partially transparent material, or a completely transparent material. The use of transparent material may be advantageous for allowing the course of healing of the wound to be checked. The use of only partially transparent or opaque material may be advantageous for sparing the patient the sight of the wound. Alternatively, the cover material (1) can also be a rigid material, which is attached over the wound region in the form of a shell opened toward the wound and is fastened in the region of the wound surrounding area (6). The cover material (1) has to be fastened in the region of the wound surrounding area (6) and sealed airtight. This can be achieved, for example, by the cover material (1) having an adhesive edge. The adhesive edge should be protected by protective strips right until application of the bandage. Alternatively, an adhesive substance can be applied to either the edge of the cover material (1) and/or the intact skin in the region of the wound surrounding area. The advantage of this is that adaptation of the cover material to the shape and size of the wound is more readily possible. Fastening and airtight sealing of the apparatus can, however, also be achieved by the use of adhesive strips or an adhesive compound.

In the embodiment shown in FIG. 1, the means for the functional connection of the wound space (5) to a negative-pressure source (10) situated outside the cover material comprises a negative-pressure connecting piece (7), which is also referred to as a port. The negative-pressure connecting piece (7) is connected to the negative-pressure unit (10) via a hose (8). Situated between the negative-pressure connecting piece (7) and the negative-pressure unit (10) is, in addition, a collection vessel (9) for the aspirated liquids. In this embodiment, the negative-pressure connecting piece (7) is situated on the outer side of the airtight cover material (1) that is facing away from the wound. To functionally connect the wound space (5) to a negative-pressure unit (10) situated outside the cover material, it is necessary in this arrangement for one or more openings passing through the cover material (1) to be situated in the region of the negative-pressure connecting piece (7). In addition, an airtight seal must be ensured. Suitable negative-pressure connecting pieces which usually comprise adhesive fasteners for attaching the port to the cover material are commercially available, for example the negative-pressure connecting piece “VivanoTec” Port from Paul Hartmann AG, Germany.

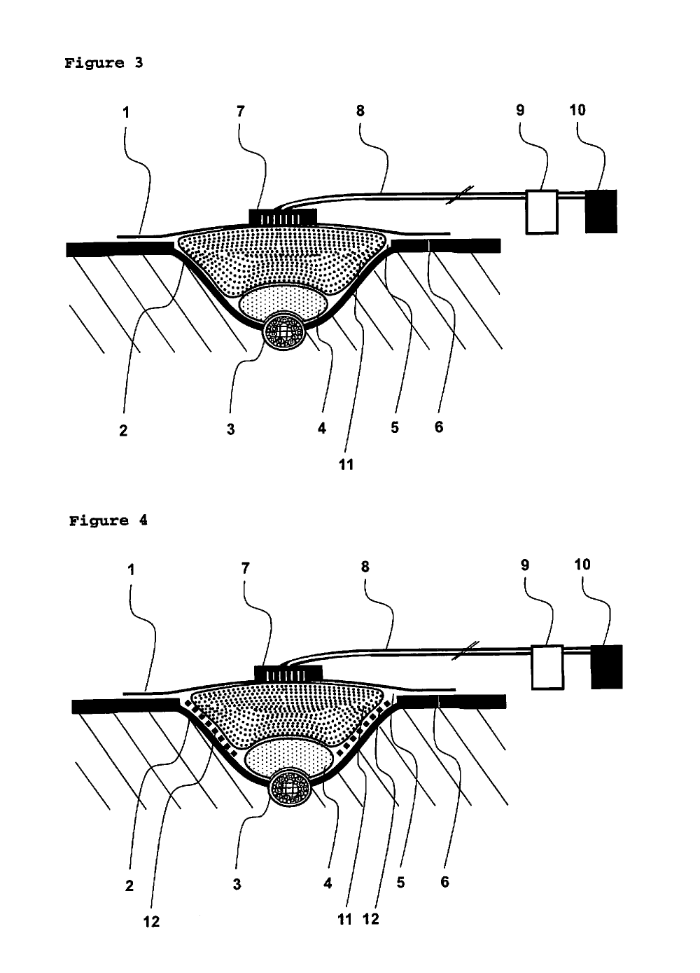

Further embodiments of the apparatus according to the invention for negative-pressure wound therapy are shown in FIGS. 2 and 3. In contrast to the apparatus shown in FIG. 1, the apparatus according to FIGS. 2 and 3 additionally comprises in both cases a pressure-distribution layer (11) which is arranged between absorbent (4) and cover material (1). According to the arrangement of bandage components which is shown in FIG. 2, the entire wound surface (2) including the exposed bone section (3) is, here, likewise substantially contacted with and covered by the absorbents (4).

According to the variant shown in FIG. 3 of the apparatus for negative-pressure wound therapy, the exposed bone section (3) has been substantially contacted with and covered by the absorbent (4), in contrast to the embodiments shown in FIGS. 1 and 2. In this variant, the absorbent (4) covers the exposed bone section (3) as completely as possible. The pressure-distribution layer (11) is applied to the absorbent (4). The wound space (5) is thus substantially completely filled owing to the pressure-distribution layer (11) which is arranged in this embodiment between the absorbent (4) and the cover material (1). In this variant, the pressure-distribution layer (11) is likewise in direct contact with that part of the wound surface (2) which does not comprise a sensitive structure, in contrast to the embodiment shown in FIG. 2. In this variant, the pressure-distribution layer (11) is thus likewise not in direct contact with the bone (3).

The apparatuses shown in FIGS. 2 and 3 both comprise only one pressure-distribution layer (11). It is also possible to use multiple pressure-distribution layers, it being possible to combine different pressure-distribution layers with one another. The additional pressure-distribution layer (11) can be designed in such a way that liquid such as wound exudate is passed therethrough. To this end, the pressure-distribution layer can contain suitable channels or a suitable opening. Alternatively, it can consist of a material which allows the passage of wound exudate without further arrangements.

A further preferred embodiment of the apparatus according to the invention for negative-pressure wound therapy is shown in FIG. 4. Here, the apparatus additionally comprises a wound contact layer (12) which is applied to that part of the wound surface (2) which does not comprise a sensitive structure. The pressure-distribution layer (11) is accordingly applied to the wound contact layer (12) and to the absorbent (4). The wound contact layer (12) should be designed in such a way that wound exudate can pass the wound contact layer (12). It is also possible to use multiple wound contact layers. In this case, it is also possible to combine different wound contact layers with one another. The wound contact layer (12) can also comprise fibers or tamponing ribbons.

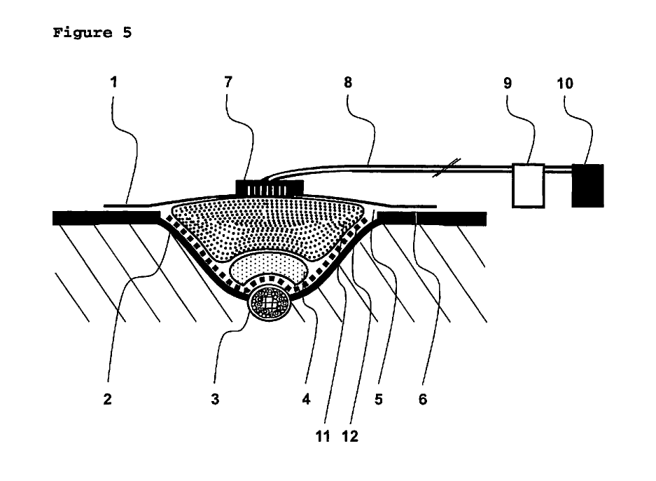

A further embodiment shown in FIG. 5 of the apparatus according to the invention for negative-pressure wound therapy differs from the embodiment shown in FIG. 4 in that the wound contact layer (12) is applied to the entire wound surface and thus also covers the sensitive structure (exposed bone section, 3). In this embodiment, it must be ensured that the wound contact layer (12) does not prevent the absorbent (4) from being able to keep the bone section (3) in a moist environment.

### Exemplary Embodiment

Wound type: Venous ulcer with exposed tendon and bone tissue

Firstly, the wound is freed of necroses and contamination (debridement). Thereafter, the region of the wound surface in which tendon and bone are exposed is completely covered with an absorbent (TenderWet® active cavity, Paul Hartmann AG, Germany). TenderWet active cavity is a wound dressing pad which is saturated with Ringer's solution and which comprises a shell composed of an interlock knit composed of polypropylene fibers and, arranged therein, an airlaid composed of a superabsorbent polyacrylate, cellulose fibers and polypropylene fibers. The rest of the wound is treated with an open-cell polyurethane foam (VivanoMed Foam, Paul Hartmann AG, Germany) as pressure-distribution layer. In this step, the foam is placed both around the absorbent and over the absorbent and the wound space is completely filled. The wound treated in this way is sealed airtight with a polyurethane film (Hydrofilm®, Paul Hartmann AG, Germany) as cover material. Then, a hole is cut into the cover material and a negative-pressure connecting piece (VivanoTec Port, Paul Hartmann AG, Germany) is attached thereover in order to ensure optimal drainage of the wound exudate. The negative-pressure connecting piece is connected via a hose to a collection vessel and a negative-pressure unit (VivanoTec negative-pressure therapy unit with secretion container, Paul Hartmann AG, Germany). On the negative-pressure unit, a negative pressure of 125 mmHg in continuous mode is set. The components used for the wound bandage (absorbent, pressure-distribution layer, cover film, negative-pressure connecting piece) and also the hose and the collection vessel are used in the sterile state.

After attachment of the bandage and activation of the negative-pressure unit, negative pressure builds up in the wound space. The wound exudate can be aspirated and collected in the collection vessel. If the collection vessel is completely filled, it can be exchanged for a new one, without the bandage having to be changed while doing so.

After 2 days, the bandage is changed and the wound assessed. After renewed debridement, a new wound bandage is applied as described previously. This is repeated until granulation tissue has sufficiently formed, and so the tendon and bone tissue is no longer exposed (assessment by physician). Thereafter, the patient is prepared for a split-thickness skin graft. Here, a thin layer of skin from an intact site of the body of the patient is taken. This is enlarged in terms of its area by several fold by means of an apparatus and fastened on the ulcerous wound as a split-thickness skin graft. Thereafter, the negative-pressure therapy is continued, the absorbent no longer being used and the wound space being completely filled with the open-cell polyurethane foam (VivanoMed Foam), optionally in combination with a wound contact layer (for example, Atrauman® or Atrauman Paul Hartmann AG, Germany). Finally, the negative-pressure therapy is ended. The wound treatment is continued with a hydrogel or hydrocolloid wound bandage (HydroTac® or Hydrocoll®, Paul Hartmann AG, Germany) until complete wound closure.

1. An absorbent body (4) comprising a superabsorbent polymer, for the therapeutic treatment by means of negative pressure of a wound comprising, at its surface (2), a tissue structure (3) which is sensitive and lies deep in the intact body.

2. An absorbent body (4) comprising a superabsorbent polymer, for the therapeutic treatment of a wound by means of negative pressure, wherein the absorbent body (4) is contacted, during the treatment, with a tissue structure (3) at the surface of the wound (2), which structure is sensitive and lies deep in the intact body, in order to protect the tissue structure (3) from drying out.

3. The absorbent body (4) according to claim 1, wherein the tissue structure (3) which is sensitive and lies deep in the intact body is a ligament, a tendon, cartilage and/or bone.

4. The absorbent body (4) according to claim 1, wherein the tissue structure (3) which is sensitive and lies deep in the intact body occupies an area of at least 1 cm².

5. The absorbent body (4) according to claim 2, wherein the absorbent body (4) is contacted with the tissue structure (3) which is sensitive and lies deep in the intact body for at least 6 hours.

6. The absorbent body (4) according to claim 1, wherein the superabsorbent polymer is a superabsorbent polyacrylate.

7. The absorbent body (4) according to claim 1, wherein the absorbent body (4) additionally comprises a mixture of cellulose fibers and polypropylene fibers as support material.

8. The absorbent body (4) according to claim 1, wherein the absorbent body (4) is an absorbent body (4) surrounded by a textile shell.

9. The absorbent body (4) according to claim 1, wherein the absorbent body (4) is charged with a solution.

10. The absorbent body (4) according to claim 9, wherein the absorbent body (4) is charged with at least 500 percent by weight, based on the weight of the dry absorbent body (4), of the solution.

11. The absorbent body (4) according to claim 9, wherein the solution is a Ringer's solution.

12. The absorbent body (4) according to claim 1, wherein the absorbent body (4) comprises an antimicrobial substance.

13. A device for negative-pressure wound therapy, comprising
a) an airtight cover material (1) for airtight sealing of the wound and of the wound surrounding area (6),
b) means (7) for the functional connection of the wound space (5) to a negative-pressure source (10) situated outside the cover material (1), making it possible to produce negative pressure in the wound space (5) and to aspirate liquids from the wound space (5),
c) at least one absorbent body (4) according to claim 1 which is introducible into the space between the surface of the wound (2) and the cover material (1).

14. The device for negative-pressure wound therapy according to claim 13, wherein the device additionally comprises at least one pressure-distribution layer (11) which is introducible between the absorbent body (4) and the cover material (1).

15. The device for negative-pressure wound therapy according to claim 14, wherein the pressure-distribution layer (11) can be open-cell or semi-open-cell foam, a spacer knit, a textile layer, a structured gel or a permeable nonwoven layer.

16. The device for negative-pressure wound therapy according to claim 15, wherein the open-cell or semi-open-cell foam is polyvinyl alcohol foam, silicone foam or polyurethane foam.

17. The device for negative-pressure wound therapy according to claim 13, wherein the device additionally comprises at least one wound contact layer (12) which is introducible between the surface of the wound (2) and the pressure-distribution layer (11) and/or between the surface of the wound (2) and the absorbent body (4).

18. The device for negative-pressure wound therapy according to claim 17, wherein the wound contact layer (12) is a polyurethane layer, a hydrocolloid layer, a structured gel, a polyorganosiloxane layer, a permeable nonwoven layer or a lattice tulle.

19. The absorbent body (4) according to claim 2, wherein the tissue structure (3) which is sensitive and lies deep in the intact body is a ligament, a tendon, cartilage and/or bone.

20. The absorbent body (4) according to claim 2, wherein the tissue structure (3) which is sensitive and lies deep in the intact body occupies an area of at least 1 cm².
